(19) United States
(12) Patent Application Publication
(10) Pub. No.: US 2025/0185905 A1
(43) Pub. Date: Jun. 12, 2025

(54) RETRACTOR MOUNTING DEVICE FOR SURGERY

(71) Applicant: UIF (University Industry Foundation), Yonsei University, Seoul (KR)

(72) Inventor: Hyung Seok PARK, Seoul (KR)

(73) Assignee: UIF (University Industry Foundation), Yonsei University, Seoul (KR)

(21) Appl. No.: 18/976,412

(22) Filed: Dec. 11, 2024

(30) Foreign Application Priority Data
Dec. 12, 2023 (KR) 10-2023-0179279

(51) Int. Cl. A61B 1/32; A61B 1/00

(52) U.S. Cl. CPC A61B 1/32; A61B 1/00133; A61B 1/00154

(57) ABSTRACT

Disclosed is a retractor mounting device for a surgery including a mounting unit, a main body provided at an end of the mounting unit, a retraction unit installed to be elevated on the main body, a sub-body disposed to be spaced apart from the main body, and an endoscope unit installed to be movable or rotatable on the sub-body.

### CROSS-REFERENCE TO RELATED APPLICATIONS

A claim for priority under 35 U.S.C. § 119 is made to Korean Patent Application No. 10-2023-0179279 filed on Dec. 12, 2023 in the Korean Intellectual Property Office, the entire contents of which are hereby incorporated by reference.

### BACKGROUND

Embodiments of the present disclosure described herein relate to a retractor mounting device for a surgery, by which a position of a retractor may be maintained at a set height or position while a doctor does not directly hold the retractor.

In general, a breast is a representative body part that symbolizes the beauty of women, and all women have a desire to maintain their breasts in a beautiful shape. Accordingly, when the breasts are sagging due to aging or are congenitally weak, or the shape is not normal due to diseases, and the like, they are being reshaped and restored to a healthier and more beautiful state through a surgery. In addition, breast cancers are the most common malignant tumors in the world, and 2.3 million new cases occur worldwide and more than 20,000 new cases occur in Republic of Korea each year.

In the case of a breast cancer surgery or breast enlargement surgery, among these breast plastic surgeries, surgeries are performed by incising parts of a body, such as an armpit, an areola, an inframammary gland, and a navel to excise the breast cancer or the breast tissue, or by inserting an implant into the breast for aesthetic purposes. Recently, breast implant insertion surgeries have been gaining attention for the purpose of further enhancing the beauty of women, and are also performed to preserve the quality of life after a mastectomy for a breast cancer treatment. In the case of external breast augmentations, the most preferred method is to incise a part of the armpit and insert the implant under the pectoralis major muscle to minimize the external appearance of the scar from the breast surgery, and in the case of the breast surgery for the breast cancer treatment, a method has also been introduced to minimize scars by making an incision in the armpit or the outer chest area covered by the arm or underwear by using a robotic endoscope.

Usually, a surgery to insert the implant under the pectoralis major muscle by incising a part of the armpit or to excise breast tumors or breast tissues requires a surgery at a distance from the chest, so it is common to use an endoscope. In the case of breast augmentations or breast reconstructions, an armpit section (3 cm to 5 cm) is first incised, then the mass thoracic muscle and the rib membrane are first peeled to a lower part of the breast by using a stripper, and the prosthesis is inserted, and in the case of mastectomy, the surgical site is removed by inserting an instrument into a similar area.

In this case, because the shape of the breast that is visible from the outside is determined depending the location, at which the implant is inserted, an endoscope is inserted to identify the internal tissues of the body, and to determine the location of the implant, an endoscopic surgical instrument, such as an endo-shear, endo-scissor, or endo-dissector is used to precisely perform a second dissection and hemostasis between the pectoralis major muscles and the costal membrane.

Meanwhile, a retractor is a surgical instrument that is located in an interior of the body to secure the field of view of a surgeon during a surgical operation, and various types of retractors are known according to surgical methods or surgical locations. In the case of a retractor for a breast augmentation, a retractor that is formed integrally with an endoscopic camera is generally used. Of course, the retractor may be used in surgeries that require a surgical space, such as a thyroid surgery as well as a breast cancer or a breast augmentation. In the conventional surgical method using a retractor, when the surgeon is right-handed, the surgeon uses an endoscopic-integrated retractor in the left hand and uses an endoscopic surgical instrument, such as an endo-shear, an endo-scissor, or an endo-dissector in the right hand to perform the surgery. However, in this case, the weight of the endoscopic camera, the weight of the retractor, and the traction force that lifts the pectoralis major muscle have been pointed out as a problem, in which fatigue accumulates in the surgeon's left hand in cases of long-term surgeries and when multiple surgeries are performed in succession.

### SUMMARY

Embodiments of the present disclosure provide a retractor mounting device for a surgery, by which a position of a retractor may be maintained at a set height or position while a doctor does not directly hold the retractor.

The tasks of the present disclosure are not limited to the tasks mentioned above, and other unmentioned tasks may be clearly understood by those skilled in the art from the description below.

According to an embodiment, a retractor mounting device for a surgery includes a mounting unit, a main body provided at an end of the mounting unit, a retraction unit installed to be elevated on the main body, a sub-body disposed to be spaced apart from the main body, and an endoscope unit installed to be movable or rotatable on the sub-body.

The retraction unit may include a retractor bent on the main body to be inserted into a body, and a mounting part disposed at a lower portion of the retractor, and on which a light or a suction unit is mounted.

The retraction unit may further include a first elevation part that elevates the retractor from the main body.

The retraction unit or the main body may include a first fixing lever that fixes a height of the first elevation part such that the first elevation part is attached to or detached from a preset height.

The main body may include a first motor, and a conversion part that converts a rotational force of the first motor to a linear reciprocal movement of the elevation part.

The sub-body may include a second motor, and a support frame extending in a horizontal direction from the main body to be coupled to pass through the sub-body.

The sub-body may include the sub-body is moved in a horizontal direction on the support frame by using a rotational force of the second motor.

The endoscope unit may include a second elevation part disposed to be elevated from the sub-body, a second fixing lever that fixes the second elevation part on the sub-body, an extension extending from an end of the second elevation part toward the retraction unit, and a ball joint mounted such that an endoscope is rotatable at an end of the extension.

The ball joint may include a shield housing fixed to an end of the extension, a ball stud coupled to be rotatable in an interior of the shield housing, and an anchor integrally extending from the ball stud and having a mounting hole such that the endoscope is mounted therein.

The ball joint may include an endoscope guide inserted into the mounting hole and that guides a position, in which the endoscope is to be inserted.

The mounting unit may include a fixing part fixed onto an installation surface, a pair of first poles extending on the fixing part in a vertically upward direction, link parts, ends of which are connected to the first poles, respectively, and selectively pivoted toward a horizontal direction, and second poles connected to opposite ends of the link parts, respectively, and a lower portion of which is fixed to the main body.

The fixing part may be provided to be movable along a guide rail fixed onto an installation surface in a horizontal direction.

Each of the first poles may include a guide groove provided in an area which one end of each of the link part contacts.

Each of the link parts may include a third fixing lever, one end of which is disposed to be movable along the guide groove, and that fixes the link part to a preset height.

The link parts may be fixed to be located at different heights on the first poles.

Each of the link parts may include a first link member, on end of which is connected to the first pole, and a second link member, one end of which is connected to an opposite end of the first link member to be rotatable, and an opposite end of which is connected to the second pole.

Each of the link parts may be pivoted in a direction, in which a first pivot area, to which any one first link member and any one second link member are connected, and a second pivot area, in which another first link member and another second link member are connected, become more distant from each other.

Specific details of other embodiments are included in the detailed description and drawings.

### BRIEF DESCRIPTION OF THE FIGURES

The summary described above as well as the detailed description of preferred embodiments of the present disclosure that will be made below will be better understood when read in relation to the accompanying drawings. The preferred embodiments are illustrated in the drawings for the purpose of illustrating the present disclosure. However, it should be understood that the present disclosure is not limited to the precise disposition and means illustrated:

FIG. 1 is a perspective view illustrating a state, in which a retractor mounting device for a surgery according to the present disclosure is installed on a bed;

FIG. 2 is a partially enlarged view illustrating right side surfaces of a retraction unit and an endoscope unit illustrated in FIG. 1;

FIG. 3 is a partially enlarged view illustrating left side surfaces of the retraction unit and the endoscope unit illustrated in FIG. 1;

FIG. 4 is a partially enlarged view illustrating front surfaces of the retraction unit and the endoscope unit illustrated in FIG. 1;

FIG. 5 is a block diagram illustrating main components of the retractor mounting device for a surgery illustrated in FIG. 1;

FIGS. 6A and 6B are reference views illustrating a state of use for a breast cancer surgery using the retractor mounting device for a surgery illustrated in FIG. 1; and

FIG. 7 is a partially enlarged view illustrating a mounting unit of the retractor mounting device for a surgery illustrated in FIG. 1.

### DETAILED DESCRIPTION

Hereinafter, preferred embodiments of the present disclosure will be described in detail with reference to the accompanying drawings. The advantages and features of the present disclosure, and a method for achieving them will become clear with reference to the embodiments that will be described in detail together with the accompanying drawings. However, the present disclosure is not limited by the embodiments disclosed hereinafter but may be implemented in various different forms, and the embodiments are provided simply to make the disclosure of the present disclosure complete and inform an ordinary person in the art of the scope of the present disclosure, and the disclosure is only defined by the scope of the claims. Throughout the specification, the same reference numerals refer to the same elements.

The present disclosure may have various modifications and embodiments, and thus specific embodiments are illustrated and described in the drawings.

However, this is not intended to limit the present disclosure to specific embodiments, but should be understood to include all modifications, equivalents, and substitutes included in the spirit and technical scope of the present disclosure.

Terms including ordinal numbers such as first, second, etc. may be used to describe various elements, but the elements are not limited by the terms.

The terms are used only for the purpose of distinguishing one element from another.

For example, without departing from the scope of the present disclosure, a second element may be referred to as a first element, and similarly, a first element may also be referred to as a second element.

The term “and/or” includes a combination of a plurality of related described items or any one of a plurality of related described items.

When a component is referred to as being “connected” or “electrically connected” to another component, it should be understood that it may be directly connected or electrically connected to that other component, but that there may be other components therebetween.

On the other hand, when a component is referred to as being “directly connected” or “directly electrically connected” to another component, it should be understood that there are no other components therebetween.

The terms used in the application are used only to describe specific embodiments and are not intended to limit the present disclosure.

A singular expression includes a plural expression unless the context clearly indicates otherwise.

When the terms, such as “comprise” and/or “comprising”, is used in the specification, it should be understood that they specify presence of the above-mentioned features, numbers, steps, operations, components, parts, and/or combinations thereof, and do not exclude presence or addition of one or more other numbers, steps, operations, components, parts, and/or combinations thereof.

Hereinafter, embodiments will be described in detail with reference to the accompanying drawings, but the same or corresponding components will be given the same reference number regardless of the reference numerals, and a repeated description thereof will be omitted.

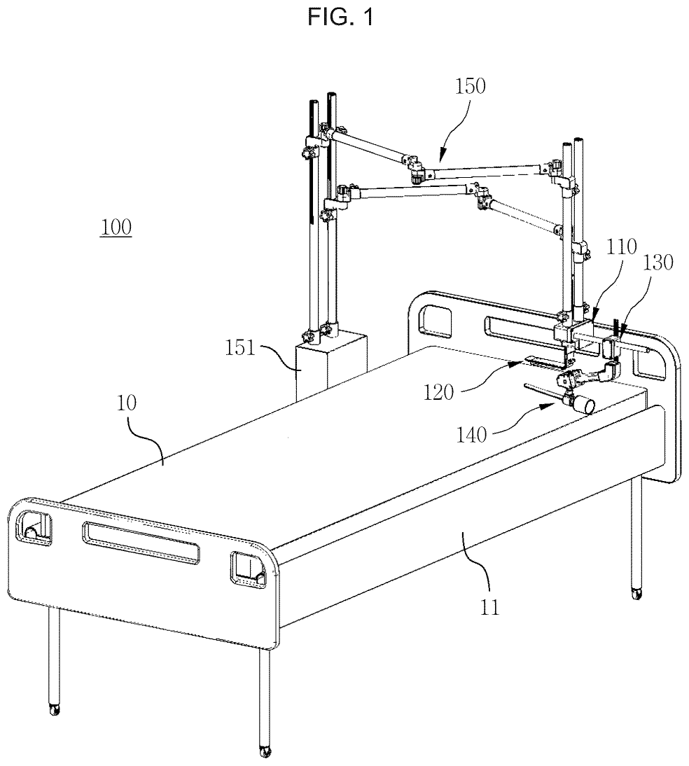

FIG. 1 is a perspective view illustrating a state, in which a retractor mounting device for a surgery according to the present disclosure is installed on a bed.

Referring to FIG. 1, a retractor mounting device 100 for a surgery according to an embodiment of the present disclosure may include a main body 110, a retraction unit 120, a sub-body 130, an endoscope unit 140, and a mounting unit 150.

First, the main body 110 may be fixed to the mounting unit 150, and may be disposed to be movable. The main body 110 has a substantially rectangular parallelepiped shape, but the shape of the main body 110 is not limited thereto.

Furthermore, the retraction unit 120 may be provided on a front side of the main body 110.

The retraction unit 120 may be disposed to be elevated from the front side of the main body 110 in an up/downward direction.

Furthermore, the sub-body 130 may be provided on a rear side of the main body 110.

The sub-body 130 may be disposed to be movable from the main body 110 in a forward/rearward direction.

Furthermore, the endoscope unit 140 may be coupled to the sub-body 130.

The endoscope unit 140 may be disposed to be elevated from the sub-body 130. Of course, the endoscope unit 140 may be moved in the up/downward direction and the forward/rearward direction because the sub-body 130 is moved from the main body 110 in the forward/rearward direction.

Furthermore, the mounting unit 150 may dispose the main body 110 in a position that is set in a surgical space, and may dispose it in a reset position when the set position is reset. The mounting unit 150 will be described as an example of being installed on the bed 10 in FIG. 1, and an installation place of the mounting unit 150 is not limited thereto. Of course, the mounting unit 150 may be installed on a wall surface or a floor surface.

The mounting unit 150 may include a fixing part 151 that is fixed on an installation surface. Here, the fixing part 151 may be installed on a guide rail 11 that is formed long along a horizontal direction on a side surface of the bed 10. Furthermore, the fixing part 151 may be disposed to be movable on the guide rail 11 in the horizontal direction. Of course, the bed 10 may be a bed installed that is in a surgery room (not illustrated).

Although not illustrated in the drawings, the fixing part 151 may include a guide jig (not illustrated) that is elastically compressed or stretched with respect to a vertical direction around the guide rail 11. The guide jig may press the guide rail 11 in the vertical direction to fix the fixing part onto the guide rail, and may be firmly maintained in a fixed state by rotating a fixing lever (not illustrated) while being fixed to the guide rail.

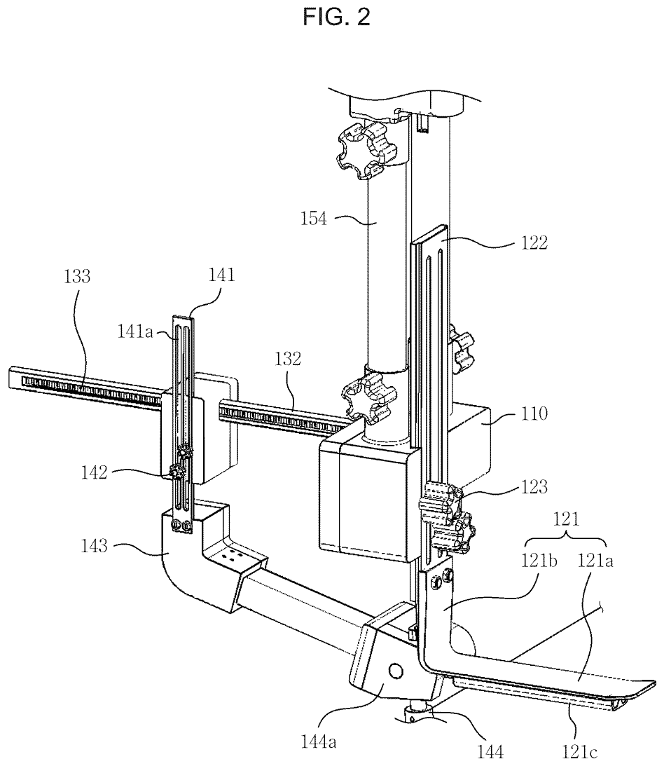

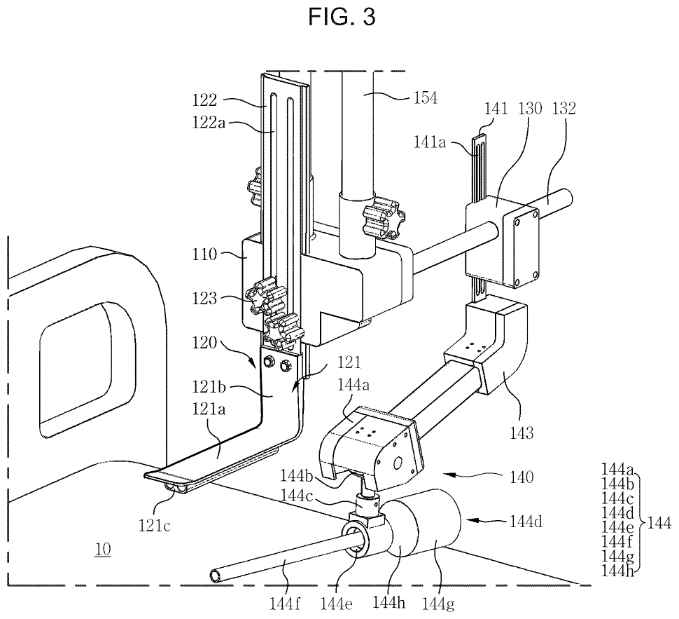

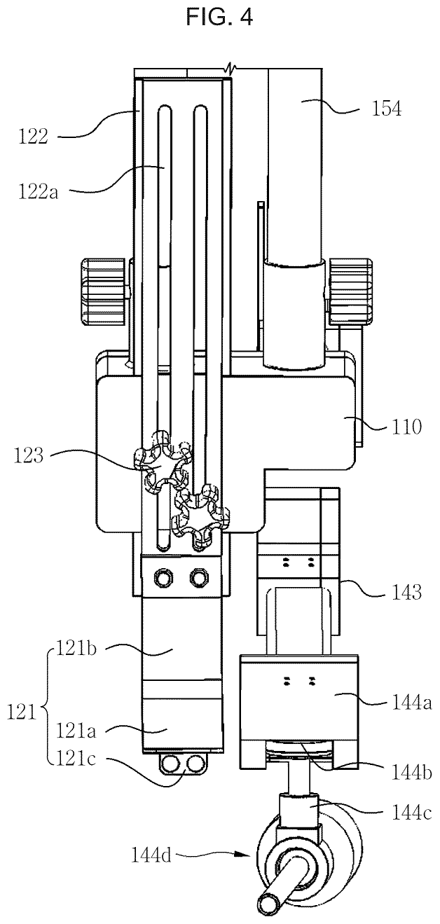

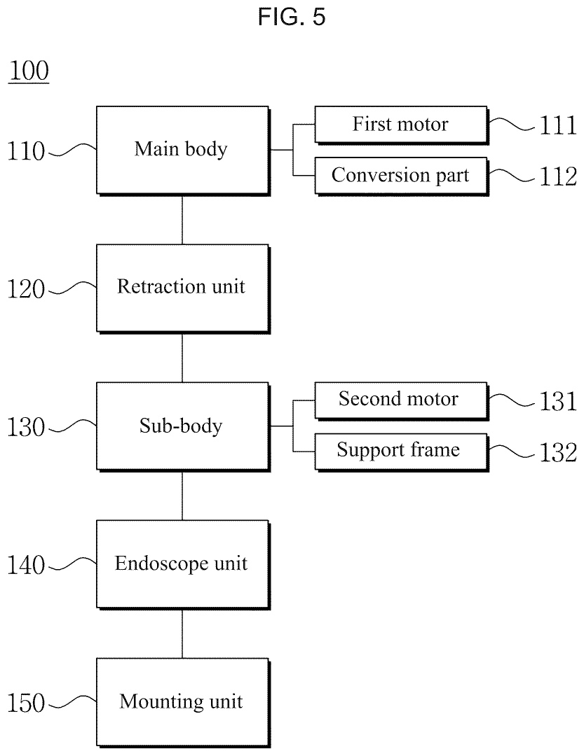

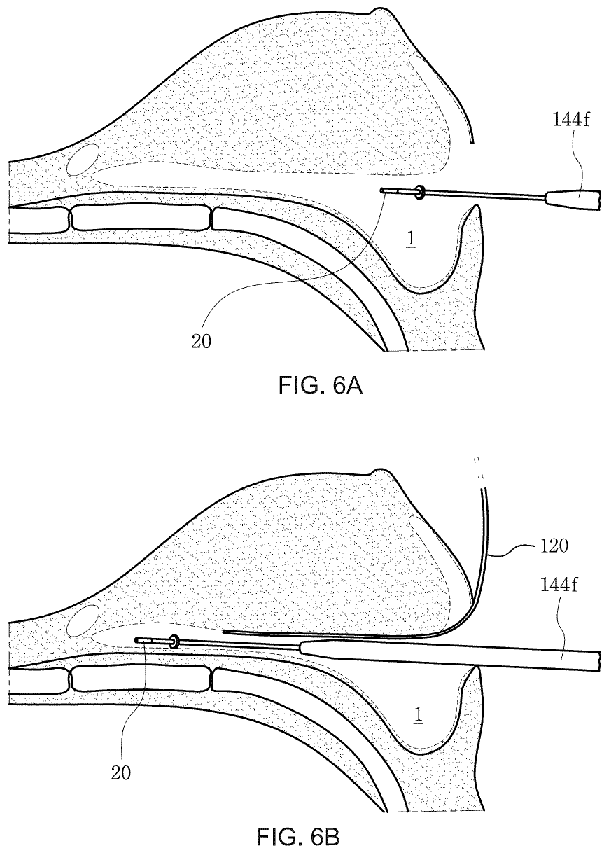

FIG. 2 is a partially enlarged view illustrating right side surfaces of a retraction unit and an endoscope unit illustrated in FIG. 1, FIG. 3 is a partially enlarged view illustrating left side surfaces of the retraction unit and the endoscope unit illustrated in FIG. 1, FIG. 4 is a partially enlarged view illustrating front surfaces of the retraction unit and the endoscope unit illustrated in FIG. 1, FIG. 5 is a block diagram illustrating main components of the retractor mounting device for a surgery illustrated in FIG. 1, and FIGS. 6A and 6B are reference views illustrating a state of use for a breast cancer surgery using the retractor mounting device for a surgery illustrated in FIG. 1.

Referring to FIGS. 1 to 5, the retractor mounting device 100 for a surgery according to an embodiment of the present disclosure may include the main body 110, the retraction unit 120, the sub-body 130, the endoscope unit 140, and the mounting unit 150.

First, the main body 110 may be fixed to the mounting unit 150, and may be disposed in a preset position. A detailed description of the mounting unit 150 will be described later.

The main body 110 may include a conversion part 112, in which a first motor 111 is mounted in an interior thereof and elevates the retraction unit 120 through a rotational force of the first motor 111.

For example, the first motor 111 may provide a rotational force, and the conversion part 112 may convert the rotational force of the first motor 111 into a linear reciprocal movement. The conversion part 112 may be implemented through mechanical elements, such as a worm and a worm gear.

Furthermore, the retraction unit 120 may include a retractor 121, a mounting part 121c, and a first elevation part 122.

Substantially, one cross section of the retractor 121 may be formed in an “L” shape. In this case, an area that protruding from a horizontal surface 121a of the retractor 121 may be inserted into the body, and an area that extends from a vertical surface 121b may be connected to a first elevation part 122.

For example, when used for a breast cancer surgery or a breast augmentation, the horizontal surface 121a of the retractor 121 may be inserted into a body through an incision area at a lower portion of the breast as illustrated in FIGS. 6A and 6B to pull a body part upward to secure a field of view of a surgical area.

Furthermore, the vertical surface 121b is maintained at a height that is set on the main body 110 so that the horizontal surface 121a may firmly support a body part. In this case, the vertical surface 121b is fixed to the first elevation part 122, and the first elevation part 122 may be coupled to be elevated from the main body 110. That is, the first elevation part 122 may be connected to the conversion part 112 of the main body 110 to be lifted or lowered, and the position of the first elevation part 122 may be fixed by a first fixing lever 123 that fixes the set height of the first elevation part 122 on the main body 110.

In this case, the first fixing lever 123 may be provided on the retraction unit 120 or the main body 110. In the embodiment, it will be described as an example that the first fixing lever 123 is mounted on the main body 110. Of course, the first elevation part 122 may be formed with a slit to be moved in the upward/downward direction around the first fixing lever 123. That is, the first fixing lever 123 installed on the main body 110 may be disposed to pass through a slit 122a of the first elevation part 122, and when movement of the first elevation part 122 is completed on the main body 110, a position of the first elevation part 122 may be firmly fixed on the main body 110 by rotating and fastening the first fixing lever 123.

Of course, the first elevation part 122 may be removed by releasing the first fixing lever 123. When the first elevation part 122 is removed in this way, the user may grip the retraction unit 120 or the first elevation part 122 to use it for a hand-held device. Alternatively, a hand-held device may be additionally attached to the first elevation part 122, or only the retractor 121 may be removed to be used for a hand-held device.

Of course, the retraction unit 120 may be moved in the up/downward direction by a rotational force of the first motor, on the main body 110.

Furthermore, the mounting part 121c may be located below the horizontal surface 121a of the retractor 121 to mount a light or a suction unit (not illustrated). Here, the mounting part 121c may include one light hole and another suction hole that are disposed in parallel. Of course, the mounting part 121c may be formed in a groove shape instead of a hole shape, but in the embodiment, a hole shape will be described as an example. Furthermore, a plurality of light holes and suction holes may be formed, respectively, and in some cases, a plurality of light or a plurality of suction units may be mounted.

Furthermore, the retraction unit 120 may be integrally formed with the retractor 121 and the mounting part 121c.

Furthermore, the sub-body 130 may be disposed on a rear side of the main body 110. The sub-body 130 may include a second motor 131 and a support frame 132.

The sub-body 130 may be mounted on the support frame 132 that extends from the main body 110 to the horizontally rear side. The sub-body 130 may be disposed to be movable in the forward/rearward direction on the support frame 132.

The sub-body 130 may be moved in the forward/rearward direction on the support frame 132 by directly or indirectly using the rotational force of the second motor 131.

Furthermore, the support frame 132 may have teeth 133 that are engaged with a gear that is coupled to a rotary shaft of the second motor 131 in an interior of the sub-body 130. Of course, although not illustrated in the drawing, the gear coupled to the rotary shaft of the second motor 131 may transmit a torque that is generated through a separate reducer (not illustrated).

Furthermore, the endoscope unit 140 may include a second elevation part 141, a second fixing lever 142, an extension 143, and a ball joint 144.

The second elevation part 141 may be disposed to be elevated from the sub-body 130. Furthermore, the second elevation part 141 may be fixed to a height that is set through the second fixing lever 142 coupled to the sub-body 130. The second elevation part 141 may be formed with an elongated slit 141a that is formed to allow the second fixing lever 142 to pass therethrough. Accordingly, the second elevation part 141 may be moved in the upward/downward direction around the second fixing lever 142, and when the second fixing lever 142 is fastened, a position of the second elevation part 141 may be fixed while being closely attached to the sub-body 130.

Furthermore, the extension 143 may extend from a lower end of the second elevation part 141 toward the retraction unit 120. In this case, the extension 143 may be disposed to be inclined downward from a lower end of the second elevation part 141 toward the retraction unit 120.

Furthermore, the ball joint 144 may be mounted at an end of the extension 143.

The ball joint 144 may include a shield housing 144a, a ball stud 144b, an anchor 144c, and an endoscope guide 144d.

The shield housing 144a may provide a function of rotatably supporting the ball stud 144b in an interior thereof while being located in a lower area of the main body 110.

The ball stud 144b and the anchor 144c may be integrally formed, and may be rotatably coupled to each other in the shield housing 144a. That is, the ball stud 144b and the shield housing 144a may contact each other spherically to guide and support rotation of the ball stud 144b in an interior of the shield housing 144a.

Furthermore, the anchor 144c may extend downward from the ball stud 144b, and the mounting hole 144e, on which the endoscope guide 144d is mounted, may be formed at a lower portion thereof. Of course, the anchor 144c and the endoscope guide 144d may be integrally formed.

Furthermore, the endoscope guide 144d may have a hollow rod bar 144f that protrudes long toward the front side, and a funnel part 144g may be provided on the rear side so that the endoscope may be guided into the rod bar 144f. Of course, one cross section of the funnel part 144g may be formed larger than that of the rod bar 144f, and an area that connects the rod bar 144f from the funnel part 144g may be formed as an inclined surface 144h, a cross-section of which gradually decreases as it goes toward the rod bar. A front tip end of the rod bar 144f may extend to be located substantially below the horizontal surface 121a of the retractor 121.

As an endoscope camera 20 is inserted into the endoscope guide 144d, the introduction of the endoscope camera 20 into the body 1 may be easily guided.

Of course, because the sub-body 130 is disposed to be movable in the forward/rearward direction, the endoscope guide 144d may also be moved in the forward/rearward direction, and may be rotated around the ball stud 144b.

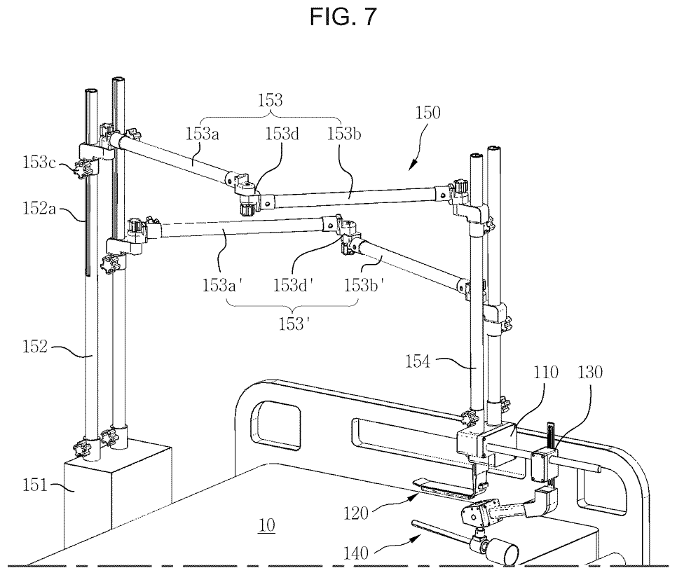

FIG. 7 is a partially enlarged view illustrating a mounting unit 150 of the retractor mounting device for a surgery illustrated in FIG. 1.

Referring to FIG. 7, the mounting unit 150 may include a fixing part 151, a first pole 152, a link part 153, and a second pole 154.

First, because a structure, in which the fixing part 151 is fixed to the bed, has been described in the description of FIG. 1, a repeated description thereof will be omitted.

Furthermore, the first pole 152 may be installed vertically upward from an upper portion of the fixing part 151. It is preferable that a pair of first poles 152 are installed in parallel to each other. Of course, the number of the first poles 152 installed is not limited thereto. A guide groove 152a may be formed in a contact area, to which one end of the link part 153 is connected, in an upper area of each of the first poles 152. Functions and effects of the guide groove 152a will be described later.

Furthermore, one end of the link part 153 may be connected to each of upper areas of the pair of first poles 152. The link part 153 may include a first link member 153a, a second link member 153b, and a third fixing lever 153c.

One end of the first link member 153a may be connected to the first pole 152.

Furthermore, one end of the second link member 153b may be rotatably connected to an opposite end of the first link member 153a. An opposite end of the second link member 153b may be connected to the second pole 154.

Accordingly, each of the link parts 153 may be adjusted such that a distance of the second pole 154 from the first pole 152 becomes larger or smaller as the first link member 153a and the second link member 153b are pivoted from each other. However, only a distance of each of the link parts 153 in the horizontal direction may be adjusted, and a height in the vertical direction may be adjusted between the first pole 152 and the first link member 153a.

The guide groove 152a may be formed in an area, in which the first link member 153a contacts an upper portion of the first pole 152. The guide groove 152a may guide movement of the first link member 153a in the vertical direction, and the first link member 153a may be fixed at a set height on the first pole 152 through the third fixing lever 153c. The third fixing lever 153c may be fixed on the first link member 153a or the first pole 152, and in the embodiment, the installation at one end portion of the first link member 153a will be described as an example in the embodiment.

Furthermore, the second pole 154 may be fixed to an upper portion of the main body 110.

The mounting unit 150 includes a pair of first poles 152, link parts 153 that are connected to the first poles 152, respectively, and second poles 154 that are connected to the link part 153 and the main body 110 therebetween, in this case, the link part 153 includes a first pivot area 153d, to which one first link member 153a and one second link member 153b are connected, and a second pivot area 153d′, to which another first link member 153a′ and another second link member 153b′ are connected, and in this case, the first pivot area 153d and the second pivot area 153d′ may be folded in a direction, in which they are spaced apart away from each other during the pivoting process. Of course, one link part 153 and another link part 153 may be mounted at different heights on each of the first poles 152. The reason why the height difference and the folding direction are different is to minimize the contact or interference of the pair of link parts 153 and 153′ with each other during the pivoting process.

Accordingly, according to the retractor mounting device for a surgery according to the embodiment of the present disclosure, the retractor may be mechanically fixed at a set height, a troublesomeness of having to hold the retractor directly may be solved, and a sharp increase in fatigue because the retractor does not have to be held directly, and the endoscopic camera, the light, and the suction unit may be mounted at the set height at the same time, and the retractor may be easily attached or detached or be changed in position by using the main body and sub-body.

Although the present disclosure has been described with reference to the embodiments, it will be appreciated by an ordinary skilled in the art, to which the present disclosure pertains, that the present disclosure may be modified and changed within the scope of the appended claims without departing from the spirits and technical field of the present disclosure. Accordingly, such modifications should also be considered to fall within the scope of the present disclosure, and the scope of the present disclosure should be determined by the claims set forth below.

What is claimed is:

1. A retractor mounting device for a surgery, comprising:
a mounting unit;
a main body provided at an end of the mounting unit;
a retraction unit installed to be elevated on the main body;
a sub-body disposed to be spaced apart from the main body; and
an endoscope unit installed to be movable or rotatable on the sub-body.

2. The retractor mounting device of claim 1, wherein the retraction unit includes:
a retractor bent on the main body to be inserted into a body; and
a mounting part disposed at a lower portion of the retractor, and on which a light or a suction unit is mounted.

3. The retractor mounting device of claim 2, wherein the retraction unit further includes:
a first elevation part configured to elevate the retractor from the main body.

4. The retractor mounting device of claim 3, wherein the retraction unit or the main body includes:
a first fixing lever configured to fix a height of the first elevation part such that the first elevation part is attached to or detached from a preset height.

5. The retractor mounting device of claim 3, wherein the main body includes:
a first motor; and
a conversion part configured to convert a rotational force of the first motor to a linear reciprocal movement of the elevation part.

6. The retractor mounting device of claim 1, wherein the sub-body includes:
a second motor; and
a support frame extending in a horizontal direction from the main body to be coupled to pass through the sub-body.

7. The retractor mounting device of claim 6, wherein the sub-body includes:
the sub-body is moved in a horizontal direction on the support frame by using a rotational force of the second motor.

8. The retractor mounting device of claim 1, wherein the endoscope unit includes:
a second elevation part disposed to be elevated from the sub-body;
a second fixing lever configured to fix the second elevation part on the sub-body;
an extension extending from an end of the second elevation part toward the retraction unit; and
a ball joint mounted such that an endoscope is rotatable at an end of the extension.

9. The retractor mounting device of claim 8, wherein the ball joint includes:
a shield housing fixed to an end of the extension;
a ball stud coupled to be rotatable in an interior of the shield housing; and
an anchor integrally extending from the ball stud and having a mounting hole such that the endoscope is mounted therein.

10. The retractor mounting device of claim 9, wherein the ball joint includes:
an endoscope guide inserted into the mounting hole and configured to guide a position, in which the endoscope is to be inserted.

11. The retractor mounting device of claim 1, wherein the mounting unit includes:
a fixing part fixed onto an installation surface;
a pair of first poles extending on the fixing part in a vertically upward direction;
link parts, ends of which are connected to the first poles, respectively, and selectively pivoted toward a horizontal direction; and
second poles connected to opposite ends of the link parts, respectively, and a lower portion of which is fixed to the main body.

12. The retractor mounting device of claim 11, wherein the fixing part is provided to be movable along a guide rail fixed onto an installation surface in a horizontal direction.

13. The retractor mounting device of claim 11, wherein each of the first poles includes:
a guide groove provided in an area which one end of each of the link part contacts.

14. The retractor mounting device of claim 13, wherein each of the link parts includes:
a third fixing lever, one end of which is disposed to be movable along the guide groove, and configured to fix the link part to a preset height.

15. The retractor mounting device of claim 14, wherein the link parts are fixed to be located at different heights on the first poles.

16. The retractor mounting device of claim 11, wherein each of the link parts includes:
a first link member, on end of which is connected to the first pole; and
a second link member, one end of which is connected to an opposite end of the first link member to be rotatable, and an opposite end of which is connected to the second pole.

17. The retractor mounting device of claim 16, wherein each of the link parts is pivoted in a direction, in which a first pivot area, to which any one first link member and any one second link member are connected, and a second pivot area, in which another first link member and another second link member are connected, become more distant from each other.
